(19) United States
(12) Patent Application Publication
(10) Pub. No.: US 2014/0194859 A1
(43) Pub. Date: Jul. 10, 2014

(54) SYSTEM AND METHOD OF PERFORMING FEMTOSECOND LASER ACCOMODATIVE CAPSULOTOMY

(71) Applicant: Pravoslava IANCHULEV, San Mateo, CA (US)

(72) Inventor: Pravoslava IANCHULEV, San Mateo, CA (US)

(21) Appl. No.: 13/834,789

(22) Filed: Mar. 15, 2013

Related U.S. Application Data
(60) Provisional application No. 61/750,841, filed on Jan. 10, 2013.

(51) Int. Cl. A61F 9/008

(52) U.S. Cl. CPC A61F 9/008
USPC 606/6

(57) ABSTRACT

Disclosed is a system and method for making a first incision in an anterior capsule of a capsular bag, the first incision being less than or equal to approximately 3.5 mm in diameter and making a second incision in the anterior capsule, the second incision being less than or equal to approximately 3.0 mm in diameter. The first incision and the second incision are positioned off-center from a center portion of the anterior capsule. The method includes performing lens fragmentation of a lens in the capsular bag to yield lens material, inserting a first instrument into the first incision, inserting a second first instrument into the second incision and removing the lens material via one of the first instrument and the second instrument and through one of the first incision and the second incision. The tensile structure of the anterior portion of the capsular bag is maintained such that accommodation exists within the eye after insertion of the intraocular lens.

### PRIORITY CLAIM

The present application claims priority to Provisional App. No. 61/750,841, filed Jan. 10, 2013, the contents of which are incorporated herein by reference

### FIELD OF THE INVENTION

The present disclosure relates to cataract surgery and more specifically to cataract surgery utilizing a laser and specially-designed instruments to perform a capsulotomy having at least one small-diameter off-center incision in the capsular bag which preserves accommodative movement transmitted by zonules and the lens capsule to the intraocular lens. Automated, computer-guided, non-manual capsulotomy in connection with automated, non-manual endocapsular lens fragmentation enable the accommodation-sparing and restoring cataract surgery.

### BACKGROUND OF THE INVENTION

A main challenge that currently exists with cataract surgery is that the surgery fails to restore the accommodation of the eye for the patient. In other words, after cataract surgery in which the central region of the anterior portion of the capsular bag is removed, the patient only maintains distance acuity rather than the ability to read or view objects close up without reading glasses. Studies show that 50% of people after cataract surgery do not see 20/20 at close distances, even with a premium accommodating intraocular lens.

FIG. 1 illustrates the basic components of the eye 100 which will be used throughout this disclosure to explain the current state of the art and the improvements disclosed herein. The lens 102 is held within a capsule or capsular bag 116 having a posterior portion or surface 104 and an anterior portion or surface 106. Connecting the capsular bag 116 to the pars plicata and pars plana of the ciliary body 114 are ciliary zonules 108. The zonules 108 function to enable the eye to focus on objects that are near or far through adjusting the tension throughout the capsular bag 116 in order to change the position and shape of the lens 102 contained within the capsular bag 116. Typical cataract surgery includes first making an incision in the cornea 112 (or the sclera) when the iris 110 is dilated. The surgeon uses the opening in the cornea 112 to perform a capsulotomy using instruments. Alternatively, a laser such as a femtosecond laser is used to cut an opening in the central portion of the anterior surface 106 of the capsular bag 116 prior to making the corneal incision. A capsulotomy in the anterior surface of the capsular bag 116 means opening a central, front portion of the lens capsule 116.

Currently, lasers are available to perform such a capsulotomy from companies such as Optimedica out of Sunnyvale Calif. Optimedica produces a laser system called the Catalys® laser which applies high resolution optical coherence tomography to capture, to a very fine degree, the three dimensional space within the eye. Based on the 3D image of the eye, the Catalys laser performs the capsulotomy and also performs fragmentation of the lens within the capsular bag using the laser to cut lens material. The lens material can be aspirated out the opening in the anterior capsular 106 through an instrument that is also positioned in the opening in the cornea. Optimedica highlights their user of an “Integral Guidance™” system that automatically identifies optical surfaces and establishes “safety zones” that only require confirmation by the surgeon to ensure that the laser pulses are delivered precisely to the desired location in the central portion of the anterior capsule. The “Integral Guidance™” system is an example of a preprogrammed approach to capsulotomy in the central portion of the anterior capsule and lens fragmentation.

Other companies such as LensX, Inc. (acquired by Alcon/Novartis), Lensar, Inc, Technolas (acquired by Bausch and Lomb), and others also produce similar laser systems for use in cataract surgery. Lasers from these companies also have the same safety zone restriction.

FIG. 2 illustrates an eye 200 and instrument 202 that performs a standard capsulotomy. The surgeon initially makes an incision in the cornea 112 having a diameter of from approximately 0.7 mm to 3 or 4 mm through which the surgeon inserts an instrument 202. The instrument is positioned through the corneal incision and onto the capsular bag 116 and lens 102. The center portion of the capsular bag 204 is typically opened with either an instrument or at least partially with the laser. Most lasers currently are programmed to identify the center portion of the eye and restrict where the laser cuts can occur in the capsular bag. Specifically, currently available lasers such as the laser from Optimedica, to insure safety of their use, restrict the positioning of the laser to cut in a central area of the eye. U.S. Application No. 2011/0022036 to Frey et al., incorporated herein by reference, illustrates such an approach where the position of the capsulotomy is always centered in the eye. Restricting the possible positions of a laser during cataract surgery to a center area of the eye for the purpose of performing a capsulotomy can prevent accidents such as tearing the anterior capsule or cutting too close to the iris 110. The approach prevents a surgeon from mistakenly positioning the laser to be off-center and thus making an incision in the wrong place that could damage the capsular bag 116 and the safety and effectiveness of the cataract operation.

The surgeon removes the portions 206 of the capsule that are cut out by the laser to open a relatively large opening in the anterior capsular of approximately 5 to 6 mm diameter 116 to enable the insertion of instruments such as an irrigation system to maintain pressure in the eye and an aspiration system to remove fragmented, hardened lens material. The fragmentation can occur either via an ultrasound device, manually, or through the use of lasers. A process called phacoemulsification (phaco) is a common technique in which an ultrasonic handpiece is equipped with a titanium or a steel tip which vibrates at an ultrasonic frequency in the range of 40 kHz. When the tip comes in contact with the lens 102, the lens is emulsified and a second instrument, sometimes called a “chopper” is used to chop up the lens such that smaller lens material pieces can be aspirated out of the eye. After removing the emulsified lens material, the surgeon inserts a synthetic intraocular lens through the opening in the cornea 112 and the capsular bag 116.

FIG. 3A illustrates a capsulotomy from a different angle. Instrument 202 is inserted through an incision 111 in the cornea 112 in order to remove the large central portion of the anterior surface 106 of the capsule 116. The cataract 102 can be fragmented in one way or another as noted above and then aspirated. A system 306 as is shown in FIG. 3B which, via a vacuum capability, sucks the lens or the lens fragments out through the opening in the capsular bag 116 after which a prosthetic intraocular lens is inserted in its place. FIG. 4 illustrates the general features of an eye 400 with the typical size of the opening in the anterior surface of the capsule which is typically in the range of 5-6 mm in size.

After cataract surgery, lens implants may have a limited accommodative movement because so much of the anterior surface 106 of the capsule 116 has been lost or cut away. Current lasers that are programmed to perform a capsulotomy restrict the available positioning of the laser to a central portion of the capsular bag 116. Thus, the conventional capsulotomy may destroy the accommodative feature because it destroys the accommodative capsular biomechanics such that accommodation is no longer possible.

### SUMMARY

Additional features and advantages of the invention will be set forth in the description which follows, and in part will be obvious from the description, or may be learned by practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth herein.

Disclosed herein are surgical methods as well as accompanying instrumentation which are developed for the purpose of performing cataract surgery while maintaining or restoring at least some level of accommodation to enable patients to focus not only far but also near. An automated, computer-guided, non-manual capsulotomy is disclosed in connection with automated, non-manual endocapsular lens fragmentation enable the accommodation-sparing and restoring cataract surgery.

Disclosed also is a minimally invasive tension-sparing capsulotomy and associated procedures and instrumentation to achieve improved cataract surgeries which preserve the elasticity of the anterior lens capsule and therefore the anatomic structure which supports the capacity of physiologic capsular accommodation. Accommodative ability is achieved by two countervailing movements: (1) movement transmitted from the ciliary muscles by the zonules 108 and the capsular bag 116 (the main portion of which is preserved by the principles disclosed herein) to the lens when the ciliary muscle relaxes for focusing in the distance; and (2) movement transmitted by the elastic memory of the capsular bag and particularly by the elastic memory of the anterior portion 106 of the capsular bag to the lens 102 when the ciliary muscle constricts for focusing at near distances. One or both of such movements can be lost after a standard cataract surgery because of the removal of the central region of the anterior surface of the capsular bag during the conventional capsulotomy.

One solution disclosed herein is to program a laser, such as a femtosecond laser or a Nd YAG laser, to generate at least one smaller off-center incision in the anterior capsular bag 106. A first incision is made in the anterior capsule and has a diameter of approximately 3 mm or less and is positioned off-center. A second optional incision is also made in the anterior capsule using the femtosecond laser (or other laser) wherein the second incision is equal to or less than 2.5 mm in diameter and also off-center. In yet another example, based on the structure of the tension sparing portions of the anterior capsule, one of the small incisions could be made in a central location. Similarly, there could also be more than one or two incisions depending on the number and location of the incisions needed to perform the cataract surgery while maintaining the accommodative feature of the capsule.

These incisions are typically circular but may also be elliptical or other shapes. The first incision and the second incision are positioned in a way so as to maintain at least a portion of the tensile strength and integrity across the anterior capsule 106. The tensile strength is maintained by strategically positioning the incision(s) away from the center portion of the anterior capsule 106. Optical coherence tomography or other imaging systems such as Scheimpflug photography, ultrasound or range-finding devices, can be used to acquire micrometer resolution data on the three dimensional images of the eye including the depth of the capsular bag over at least one of the anterior and posterior regions, or other regions of the capsular bag 116. Based on the tomography or other imaging data of at least the anterior capsule 106, the laser can be programmed to identify the optimal position of at least one off-center incision in order to perform the capsulotomy in such a way as to maintain accommodation by preserving the majority of the anterior capsule 106.

It is preferable that a femtosecond laser be programmed in order to cut with precision the incision(s). Robotic systems can be employed to perform the incision(s) as well. Such robotic devices can scan the eye in order to receive, via optical coherence tomography or other mechanism, a 3D image of at least a portion of the capsular bag. The 3D image can be used to guide a system to robotically irrigate, aspirate, and polish surfaces within a safe 3D region inside the capsular bag. U.S. Pub. No.: US 2012/0253332 A1, by Frederic Moll, references some general robotic instrumentation which can be incorporated herein for the purpose of controlling and executing the procedures disclosed. This publication is incorporated herein by reference. The surgeon or robot can safely move a tip of an instrument for irrigating and aspirating without damaging a posterior portion of the capsular bag as well as for polishing any portion of the capsular bag following the extraction of the lens material.

The system also can perform an external lens endocapsular lens fragmentation. The lens 102 in the capsular bag 116 can receive a pretreatment in order to prepare for removal of the lens material through one of the first incision and the second incision. An example system which can be utilized for lens fragmentation is disclosed by Blumenkranz et al., Pub. No. 2006/195076, incorporated herein by reference. These components can be guided by computer systems to achieve a non-manual capsulotomy that is only possible with the incision size and precision that can be done non-manually. Further, the system can perform a non-manual automated endocapsular lens fragmentation (which includes dissolution, emulsification, etc. to prepare the lens for aspiration) to enable the accommodation-sparing/restoring cataract surgery.

Novel instruments are also disclosed in order to functionally operate in cataract surgery in which minimally invasive incisions are used such as an approximately 1 mm incision in the cornea (but larger corneal incisions can also be done) followed by an incision in the capsular bag that is under approximately 3 mm of less and off center. A first instrument is inserted into the first incision in which the first instrument has a tip that is flexible and/or extendable to allow intraocular directional angulation within the capsular bag 116. The freedom of movement from a tip portion of the instrument within the capsular bag 116 is necessary because the size of the hole in the capsular bag 116 is much smaller than the incision made in a traditional cataract surgery. The improved instrumentation is necessary to enable the surgeon to move around within the chamber of the capsular bag and remove all of the fragmented lens material, as well as performing other operations such as irrigation and polishing. Note that the small opening in the cornea in addition to the small incision in the capsular bag result in two points through which the instrument must pass which restricts the available movement within the capsular bag absent the ability of a tip portion of the instrument being flexible and/or extendible.

A second instrument can be inserted into the second incision. The second instrument can include a tip that is flexible and/or extendable to allow directional angulation within the capsular bag or can be a fixed instrument such as the traditional “chopper.” The lens material is removed through at least one of the first incision and the second incision, the anterior capsule (and possibly the posterior capsule) is polished and the conclusion of the surgery is implanting a new lens through at least one of the first incision and the second incision.

Various embodiments are disclosed herein in relation to the particular steps set forth above. A first embodiment relates to a surgical procedure as generally outlined. A second embodiment involves performing a surgical procedure using at least one device including a femtosecond laser (or other type of laser) programmed in order to make the particular incisions small enough in diameter and positioned at the appropriate place. Yet another embodiment includes instrumentation which is particularly suited for performing cataract surgery when the opening in the capsular bag is much smaller than are used in traditional surgeries. Other embodiments also include particular intraocular lenses which are specifically tailored for the purpose of being inserted into a smaller incision in the capsular bag than is used in standard cataract surgeries.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only exemplary embodiments of the invention and are not therefore to be considered to be limiting of its scope, the invention will be described and explained with additional specificity and detail through the use of the accompanying drawings in which:

FIG. 1 illustrates the basic components of the eye that are relevant to cataract surgery;

FIG. 2 illustrates a prior art capsulotomy;

FIG. 3A illustrates a prior art approach of excising the anterior capsule;

FIG. 3B illustrates aspirating the lens in a prior art cataract surgery;

FIG. 4 illustrates a prior art opening in the anterior surface of the capsular bag in a capsulotomy;

FIG. 5 illustrates the positioning and relative size of incisions in the capsular bag according to an aspect of this disclosure;

FIG. 6 illustrates a method first embodiment;

FIG. 7A illustrates two off-center incisions in the anterior capsular;

FIG. 7B illustrates other shapes which can be used for incisions in the capsular bag;

FIG. 8 illustrates a side view of the capsular bag and lens;

FIG. 9 illustrates a side view showing irrigation and aspiration using two off-center incisions in the anterior capsular bag;

FIG. 10A illustrates a second embodiment;

FIG. 10B illustrates a component of the second embodiment;

FIG. 11 illustrates exemplary instrumentation according to an embodiment;

FIG. 12 illustrates a cross sectional view of an instrument in the capsular bag having the ability to flex and extend;

FIG. 13 illustrates an extendible tip on a cataract surgery instrument;

FIG. 14 illustrates another aspect of an extendible tip for cataract surgery;

FIG. 15 illustrates yet another aspect of an extendible tip for cataract surgery;

FIG. 16 illustrates a laser and/or optical coherence tomography device according to an embodiment of this disclosure;

FIG. 17 illustrates a method embodiment;

FIG. 18 illustrates another method embodiment; and

FIG. 19 illustrates a robotic embodiment and a cross sectional view of a cataract surgery using a robotic instrument.

### DETAILED DISCUSSION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

The present disclosure focuses on several embodiments, each of which relate to new surgical procedures, methods, systems, computer-readable media, and instrumentation associated with cataract surgery. The primary novelty disclosed herein relates to performing a capsulotomy by cutting at least one, and preferably two, small off-center holes in the anterior capsular bag. Such a cut can maintain the accommodation feature of the capsular following cataract surgery which can result in the patient being able to focus both near and far. Current procedures either manually or using laser technology, require cutting a larger hole in the anterior capsular bag which, while making irrigation and aspiration easier, results in a loss of accommodation and the possibility of an increase in the likelihood of posterior capsular opacification. A second embodiment covers a novel laser system. The disclosed systems provide an automated, computer-guided, non-manual capsulotomy that is performed in connection with an automated, non-manual endocapsular lens fragmentation to achieve a system and method that results in an accommodation-sparing and restoring cataract surgery.

The advent of the capsular sparing surgery disclosed herein with minimally invasive capsulotomy renders it more difficult to surgically remove the cataract and lens fragments with existing opthalmic instrumentation because that instrumentation is rigid and does not allow for directional flexing or extension when inside the eye. Existing conventional intraocular instruments such as phaco probes, irrigation probes, aspiration probes, and suction probes are able to work within the eye only by advancing, retracting or pivoting the entire instrument at a single intraocular entry-point at the level of the cornea. Such is not possible given the position and size of the incision(s) disclosed herein. Thus, a third embodiment relates to instrumentation which enables for a flexible and extendible tip for use in at least one smaller, off-center incision in the anterior capsular bag. A fourth embodiment relates to a robotic approach to performing the surgery. A fifth embodiment relates to particular intra-ocular lenses which are tailored to be inserted in through the smaller incisions contemplated herein. Furthermore, even given the accommodative saving approach disclosed herein, there will be some level of change to the tension forces between the zonules and across the capsular bag after the surgery. Since the different “host” environment for a new intraocular lens will have a particular structure, the fifth embodiment also covers lenses which are designed to factor in the changed biomechanical parameters in the environment and thus exploit or better utilize the tension forces available in the eye after surgery.

Yet another embodiment relates to a computer-readable storage medium storing instructions for controlling a computing device, which can include a laser, to perform at least a portion of the cataract operation. These various embodiments each address the disclosed benefit of performing a tension-sparing capsulotomy in connection with a non-contact laser pre-treatment of a cataract. In one aspect it is not limited to non-contrat approaches in that this disclosure contemplates systems that could be in contact with the eye. These approaches facilitate lens material extraction through the small incision capsulotomy. The success of a tension-sparing and accommodation-restoring cataract surgery is the ability to perform fragmentation and removal of lens material through the smaller incisions as shall be disclosed herein.

### First Embodiment

### Method of Performing Cataract Surgery

Phacoemulsification typically requires the surgeon to perform an anterior continuous curvilinear capsulotomy (or capsulorhexis) to create a round and smooth opening in the anterior lens capsule 106 through which the lens nucleus can be emulsified and aspirated followed by insertion of the intraocular lens implant. The main challenge to this current way of performing phacoemulsification is that the lens implants only have limited accommodative movement. A primary component to the present disclosure is the ability of performing cataract surgery while maintaining accommodation because the tensile portion of the capsular bag is preserved.

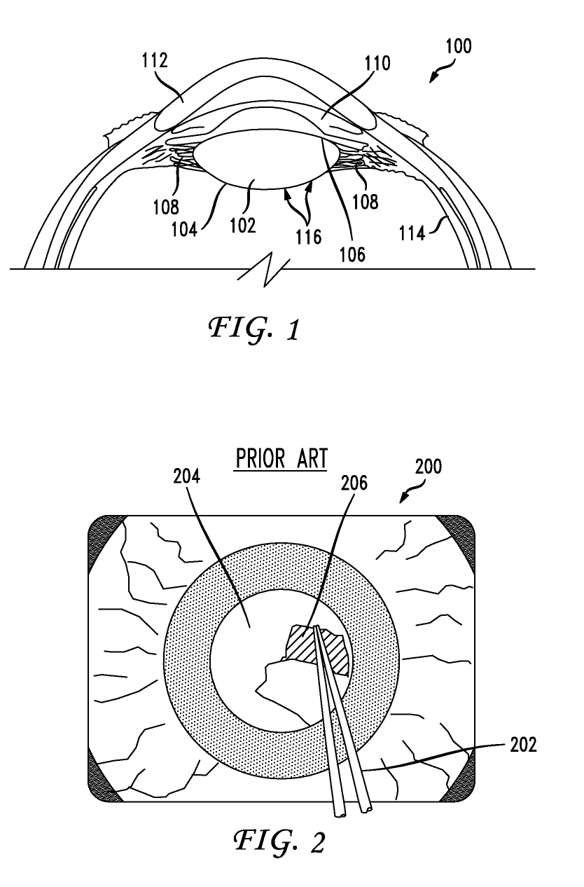

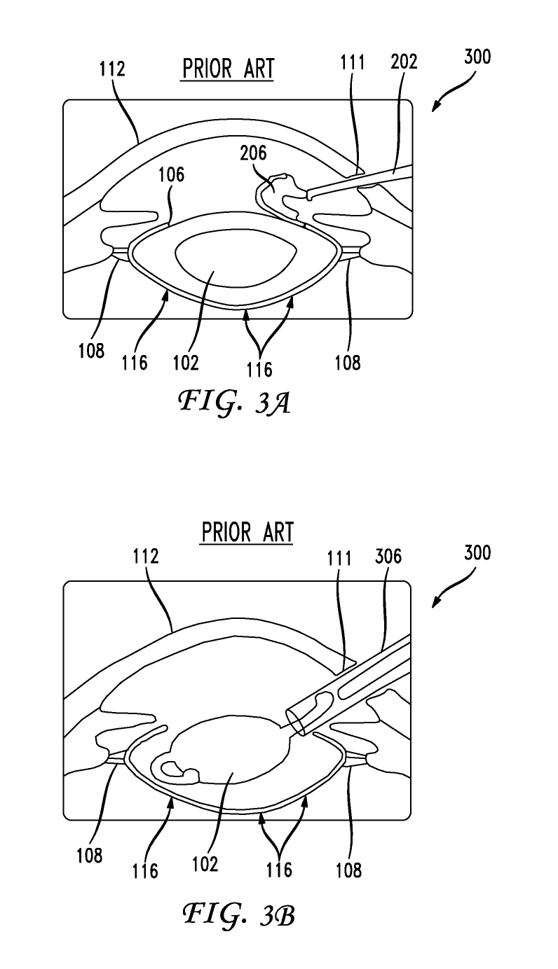

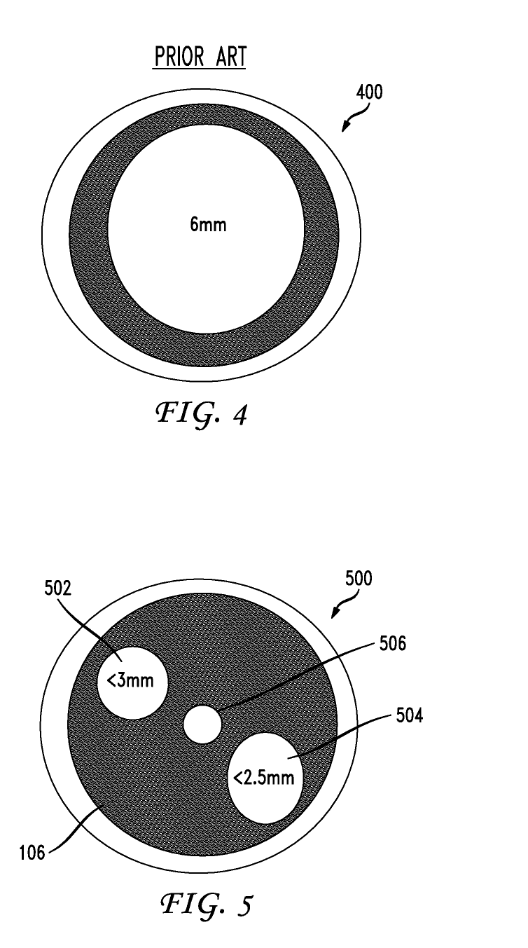

As is shown in FIG. 1, the capsular bag 116 envelops the entire lens 102 and connects it to the zonules 108. The capsular bag 116 via its tensile properties preserves the biomechanical properties of the eye as the person ages. The elastic memory of the lens capsule “molds” the compliant lens material into an accommodative conformation when the tension from the zonules is relaxed upon contraction of the ciliary muscles to shift the focus of the user to near. Similarly, lens capsule 116 captures and transmits the disaccommodative tension from the zonules 108 when the ciliary muscle relaxes which moves or adjusts the lens 102 and thus “molds” the compliant lens 102 in order to shift the focus of the user to distance. The first embodiment of the present disclosure is a new method of performing cataract surgery that does not eliminate the central core of the anterior lens capsule 116 which is usually completely exercised during a capsulotomy as is shown in FIG. 4. The surgical procedure preserves the elastic memory and therefore the capsular accommodative response, thus allowing the eye to focus at different distances and focal points after implantation of the intraocular lens.

The anterior capsule 106 of the capsular bag 116 has some particular features which are exploited in the surgical procedure and associated instrumentation disclosed herein. The anterior capsule 106 drives the tensile movement of the lens 102 and has a much higher modulus of elasticity than the lens 102 itself. The accommodative elastic forces of the capsule aid in transmitting movement from the zonules ultimately to the lens 102. The anterior capsule 106 is approximately 4× the thickness of the posterior capsule 104. The anterior capsule 106 strength properties are often wholly preserved as the patient ages. Therefore, performing the surgical procedure in connection with the disclosed instruments on people that are older in age is still beneficial.

Studies have shown that a healthy human capsule 116 can accommodate 16 diopters of accommodation. While performing the cataract surgery, as disclosed herein, may not maintain a 16 diopter accommodation, with a tension sparing capsulotomy, a goal is to maintain at least 2 to 3 diopters of accommodation even from a monofocal intraocular lens. The automated, computer-guided, non-manual capsulotomy in connection with automated, non-manual endocapsular lens fragmentation can result in a better maintenance of tension and structure in the anterior capsule after surgery. By reducing the damage to the capsule, the surgery enables an accommodation-sparing and restoring cataract surgery.

FIG. 5 illustrates an eye 500 having a first opening 502 in the anterior capsular 106 approximately equal to or less than 3 mm in diameter and a second hole 504 in the anterior capsular 106 having a diameter of approximately equal to or less than 2.5 mm. Generally, the term “approximately” means within 0-0.5 mm of range. Alternatively, the capsulotomy opening or openings may have a diameter or diameters in the range of 1.0 mm to 2.0 mm. The central portion of the anterior capsular bag 106 of the eye 500 represents the anterior surface of the capsular bag 106. As can be appreciated, much more of the capsule remains following such a surgical procedure which can preserve the central tension forces of the capsule 106. The approach disclosed herein is designed to leave more than 50% of the capsule integrity intact.

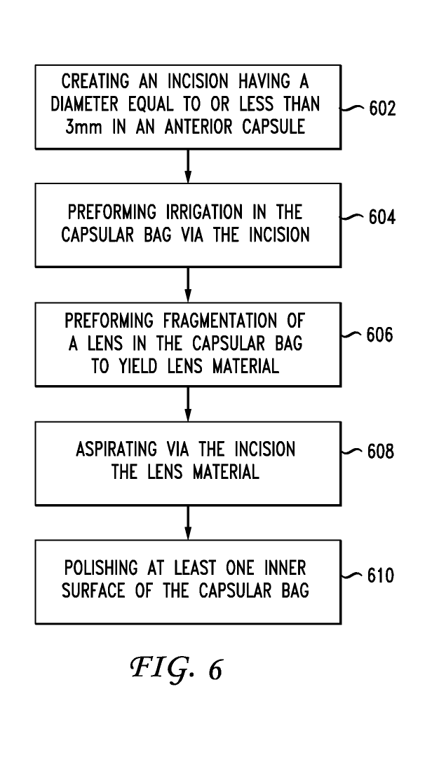

FIG. 6 illustrates a basic surgical method embodiment. This embodiment is described independent of any system such as a laser and thus could be accomplished in part or in whole manually. It is preferred that at least part of this procedure, such as step (602), would be performed in connection with a novel laser system as is disclosed herein in the second embodiment.

The method includes creating an incision having a diameter equal to or less approximately than 3 mm in an off-center position of the anterior capsule 106 of a patient (602). FIG. 5 feature 502 and 504 each provide examples of positioning and size of an incision. Generally, the methods disclosed herein are automated and computer guided. Feedback on eye structure and position during surgery causes a computer-guided system to perform the non-manual capsulotomy with computer aided decisions on number of incisions, location of incisions, size and shape of incisions.

The shape of the incision(s) can be circular, eccentric, elliptical, random, or chosen specifically based on data such as locations within the capsular bag 116 that have more or less tensile strength than other locations. In one aspect, two incisions are made so that two instruments can be utilized to irrigate the eye, aspirate lens material, and insert a replacement inter-ocular lens. Optical coherence tomography (OCT) or other imaging systems can provide a micrometer resolution and three dimensional image that can include data about the anterior capsule 106. OCT or other imaging systems data can also identify tensile strength regions and preferable locations in which to make the incision that reduce the loss of accommodation. The OCT or other imaging systems data can be used to choose a shape for the incision (2). In another aspect, an incision 506 can be made in the center or in a central region of the anterior capsule 106. The size of this incision is also preferably equal to or less than approximately 3 mm. An incision in this location could be used for the purpose of maintaining the tensile strength across the anterior capsule 106. For example, if the structure of the anterior capsule 106 indicates that the stronger tensile portions run above and below the central region of the anterior capsule 106, it can be beneficial to utilize an incision in the central region as shown in FIG. 5.

Next the method includes performing an irrigation operation in the capsular bag 116 through the incision (604). Performing irrigation in cataract surgery helps to maintain the appropriate pressure in the eye. In one aspect, a robotic system monitors and modifies where necessary the intracapsular pressure during irrigation. The monitoring and modification could also be performed manually. The method also includes performing fragmentation of a lens 102 in the capsular bag 116 to yield lens material (606). In one aspect the fragmentation is an endocapsular lens fragmentation via a laser which yields the lens material. Given that the incision in the capsular bag 116 is smaller than has been used for cataract surgery in the past, and since larger lenticular fragments will be difficult to extract through the smaller openings, part of this disclosure includes endocapsular complex, contact or non-contact lens fragmentation and/or softening and/or pre-treatment of the cataract lens in the capsular bag. The lens can also be dissolved or liquefied using known techniques.

The method includes aspirating via the incision (or a second incision) the lens material (608) and (optionally) polishing at least one inner surface of the capsular bag 116 (610). The endocapsular fragmentation, softening, and/or pre-treatment prepares the lens material for extraction through the smaller minimally invasive anterior capsular incision. Other steps include inserting an intraocular lens in place of the cataract through the incision.

Because this aspect of the disclosure relates to utilizing incisions in an off-center position from a central portion of the anterior capsular, the choice of how to perform fragmentation can be driven by the positioning of the incision. For example, the laser or other instrumentation may be programmed or used such that the size of particular pieces of lens material can be relative larger or smaller in the area of the incision. This can be based on making the aspiration easier.

The method and other embodiments preserve preferably at least one structural cross connection in the central capsular plane (for example in the central 6 mm capsular area) to maintain enough capsular tensile strength and integrity so as to enable the patient to have a higher degree of accommodation. One or more of the steps of the method are computer-aided or computer guided such that they are not manually performed. After the capsulotomy and fragmentation, one or more steps such as aspiration and irrigation, can be at least in part manually performed.

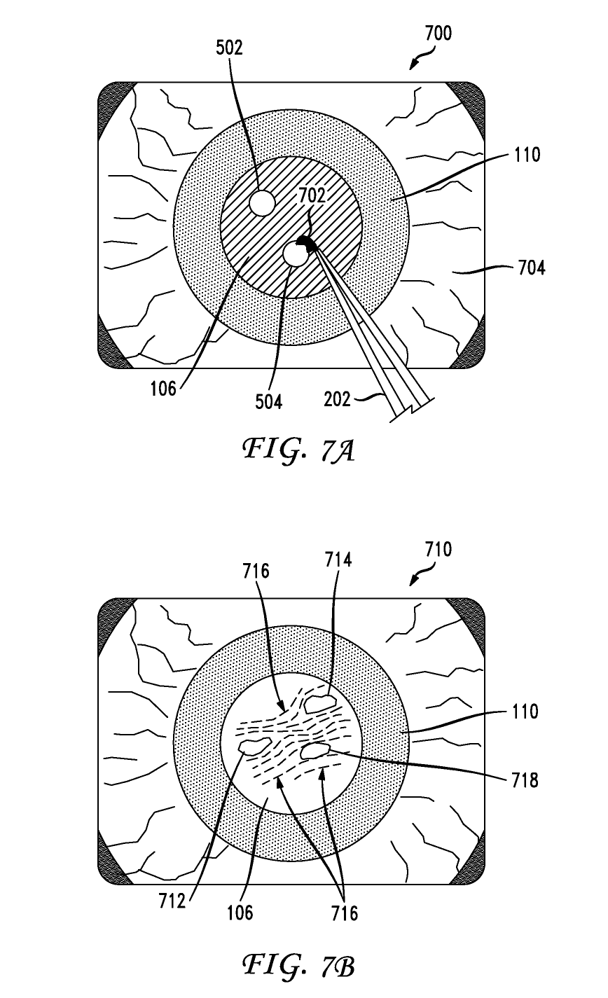

FIG. 7A illustrates creating the incision or incisions in the anterior capsule. Shown is a close up view of the eye 700 including a dilated iris 110 and the whites of the eye or the sclera 704. Shown is the anterior capsule 106 having a first incision 502 and a second incision 504 with a portion 702 of the incision 504. Also shown is an instrument 202 that is used to pull away the portion 702 of incision 504. The positioning of incisions 502, 504, 506 can be made to maintain at least one structural cross-connection in the central capsular plane which preserves the capsular tensile strength and integrity.

FIG. 7B illustrates an example 710 of not only the choice of position for incisions in the anterior capsule but also a choice of shape. The method can include making what appears to be an arbitrarily-shaped incision. Information about the anterior capsule and where cross-connection structures exist can drive a particular shape of an incision. FIG. 7B shows a structure cross-connection 716 across the anterior capsule 106. The data about the cross-connection structure can be obtained in any fashion including OCT. The representation of the data 716 in this case means where the accommodative movement is processed by the capsular bag 116 to adjust the vision of the eye. Incision 712 is at the left side and has an elliptical shape. This shape could be chosen because the tensile strength was stronger above and below that region based on the data and thus a flatter, elliptically-shaped incision was preferable.

Other techniques for gathering data regarding the characteristics of the capsular bag 116 can also be utilized. For example, one approach is discussed in the article Regional mechanical properties and stress anaylsis of the human anterior lens capsule, by R. M. Pedrigi et al., published by ScienceDirect, Vision Research 47 (2007) 1781-1789, incorporated herein by reference. In this article, a study of the mechanism of accommodation in terms of the interactions with the constituent tissues is aided by biomechanical modeling to obtain accurate measurements of the tissue mechanical properties in order to predict stresses and strains across the anterior capsule. The capsule encapsulates the lens nucleus and cortex and mediates tractions imposed into the lens by the ciliary body. The study uses linearized finite state analysis to reveal and estimate stresses and other biaxial mechanical testing methods and finite element models to generate more realistic predictions of the capsular behavior. Such studies and future experiments can provide useful data for use in the present disclosure for many purposes, including, but not limited to: (1) determining the location, number, size and/or shape of incisions in the capsular bag (anterior or posterior); (2) designing or choosing a particular IOL; (3) determining which incision based on location and/or size to insert the IOL after the capsulotomy; or (4) any other feature, device or procedure associated with the cataract surgery.

The discussion returns to FIG. 7. Incision 714 is more arbitrarily shaped. Assume in this case also that the integrity or structural cross-connection in that region was strong around the shape shown and thus the incision was made (by the surgeon or a device or programmed laser) to fit within a less valuable region defined by the opening 714. In this manner, the incision(s) is made in a more strategic manner when making incisions in the capsular bag 116. The process of dissolving, liquefying, fragmentation, softening, etc. can also take into consideration the positioning and shape of the incisions 712, 714. For example, if the incisions are not circular but are more elliptical or arbitrary shaped, then the pre treatment can cause resulting lens material to have shapes that are more easily removed from the particularly shaped hole. Thus, manually or in a programmed mode, the shape of lens material for extraction can be based on data about at least one of the position and shape of an incision in the capsular bag 116. Note that the term “arbitrarily” shaped can mean that the shape is not round or elliptical. However, the particular shape can be chosen based on the tensile structure of the anterior capsule 106 and thus which is may appear arbitrary, the shape can be chosen to follow tensile contours or structures in a way to maintain as much tensile structure across the capsule as possible or as is desirable.

Note that while it is preferred that one or two incisions are made off center in the anterior capsule, FIG. 7B also illustrates a (generally) centrally located incision 718 which can also be the sole incision and/or a secondary or third incision in the anterior capsule. The reason a central incision might be utilized is that once the structure and location of tensile saving portions of the anterior capsule 106 are known, the optimal or beneficial location of an incision can include the central portion or within the central region of the anterior capsule 106 for the purpose of maintaining as much accommodation as possible.

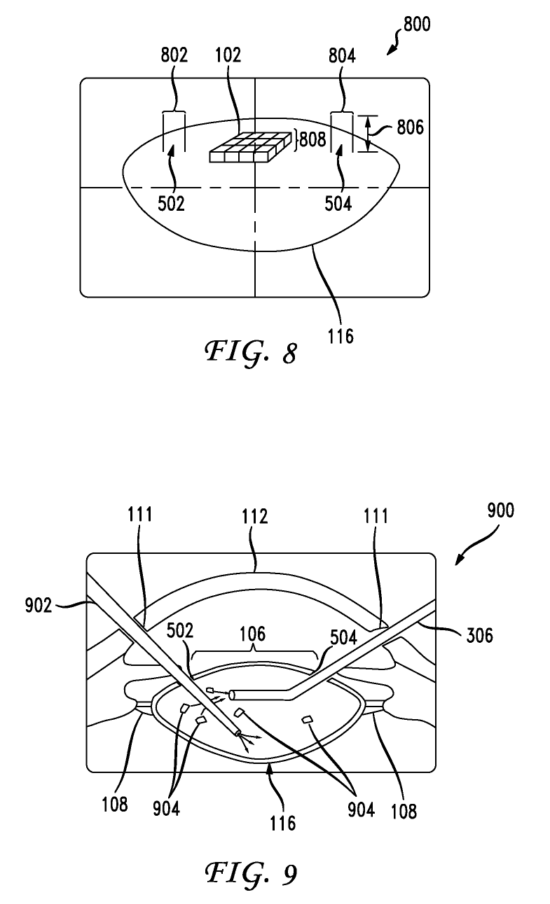

FIG. 8 illustrates a cross sectional view of the capsular bag 116 and a fragmented portion 102 of the lens. Cuts in the anterior capsular are shown as features 802 and 804 to yield incisions 502, 504, respectively. The incisions can be made manually by a surgeon or via a laser that is automated or manually handled. The height 806 illustrates a feature of how the laser would achieve the incision. The laser could be used to make the incision by sequentially or simultaneously focusing light at different depths along the path shown at feature 806. Pub. No. 2006/0195076 by Blumenkranz et al., incorporated herein by reference, illustrates some of the basic concepts for generating incisions in the capsular bag using a laser. By making cuts along the perimeter of each incision 502, 504, the laser can achieve a clean and smooth perimeter around the opening.

Because the incisions in this disclosure are small (i.e., equal to or less than approximately 3 mm), an important feature is the pretreatment of the lens to be removed such that smaller or more strategically shaped pieces are made prior to aspiration. Manually or via the use of a laser, the lens 102 can be fragmented 808 to yield lens material in small enough pieces that can be aspirated through an opening 502 or 504. As noted above, in one aspect, the surgeon may create one hole in the capsular bag 116 or may create two or more incisions depending on factors such as the structure of tension in any location of the capsular bag 116, the size of instruments, the position of the lens material, etc. As noted above as well, pretreatment should be non-contact (or can be in contact as well) and thus whether the treatment is softening, fragmentation, dissolution, or liquefaction, it should be done to enable smaller pieces of lens material to result which can be removed through a smaller incision. In one aspect, the shape of the lens material fragments is chosen based on at least one of a position, a size and a shape of the incision(s) through which the lens material must be aspirated.

FIG. 9 illustrates a cross-section of the eye 900 during cataract surgery. Openings 502, 504 have already been made in the capsular bag 116 that preserve the main central portion 106 of the anterior capsule. It is assumed at this stage that the fragmentation (and/or dissolution, liquefaction, softening, etc.) has occurred and that lens material 904 is contained within the capsular bag 116. Instrument 306 is inserted into the opening 111 in the cornea (or could be inserted through the sclera) and through the small opening 504 for aspirating the lens material 904. Another instrument 902 is shown for irrigation purposes or could be used also for chopping. The irrigation instrument 902 could also be unified with aspiration instrument 306. By maintaining the integrity of the anterior capsule 106, the cataract surgery enables the zonules 108 to continue to enable accommodative ability after the lens material 904 is aspirated and a new intraocular lens (not shown) is positioned in the capsular bag 116. As can be seen, the region 106 is not removed and thus the structural cross-connection in the central capsular plane is preserved to maintain the tensile strength and integrity across the region 106.

Alternate approaches to the method described above include determining that a single, larger incision can be made in the capsular bag 116 and that is off-center. For example, FIG. 5 shows two incisions 502, 504 each of around 3 mm or less. In another aspect, the conditions (i.e., structure and location of the structural tension in the capsular bag 116 that can be preserved to maintain accommodation) of the capsular bag and/or other factors such as type of cataract, size and shape of the lens 102, etc. could result in a preferable approach of only creating a single, off-center opening that is the same generally size as openings 502, 504 or it may be larger such as between 2.5 mm and 6 or 7 mm. The key differentiator from previous art in this case is the positioning of the capsulotomy incision is purposefully not centered in the anterior lens capsule of the eye.

### Second Embodiment

### Laser System for Performing Cataract Surgery

Femtosecond lasers have been used for performing a capsulotomy. Such femtosecond lasers allow for external lens fragmentation and pretreatment for minimally invasive removal of the lens material. Lasers which can be used when reprogrammed for the particular applications herein include the laser disclosed in Pub. No. US 2011/0245814 A1; Pub. No. US 2011/0022036 A1; and Pub. No. 2006/0195076 A1. The content of each of these applications is incorporated herein by reference. The particular manner in which incisions are made within the capsular bag 116 is not a main focus of this disclosure. Incisions can be made with any type of laser, other cutting device, or even manually. Rather, because existing laser systems are currently programmed to limit the positioning of the capsulotomy to the center of the eye, a novelty of the laser system and robotic, computer-guided control, as disclosed herein is to change the programming and thus the restriction on current systems to enable the creation of off-centered, smaller incisions in the lens capsule. Thus, an automated, computer-guided and thus non-manual capsulotomy can be achieved using a programmed laser which must change the conventional restrictions which currently require a large central incision. The computer-guided system disclosed herein will location one or more smaller incisions that are positioned in order to maintain the tensile structure across the anterior (or posterior) capsulary 106. The choice of incision location may be central but can also be off-center.

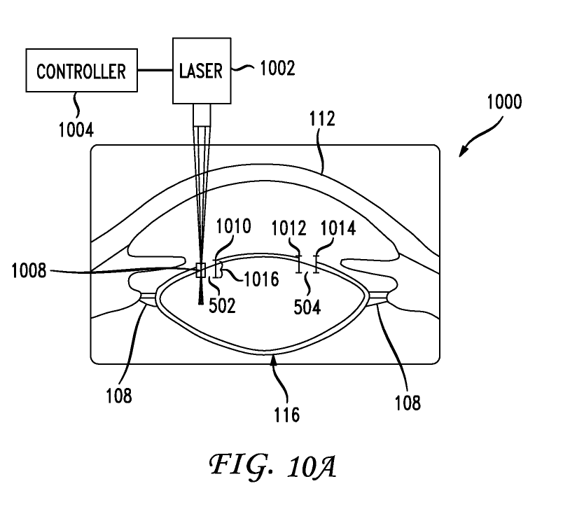

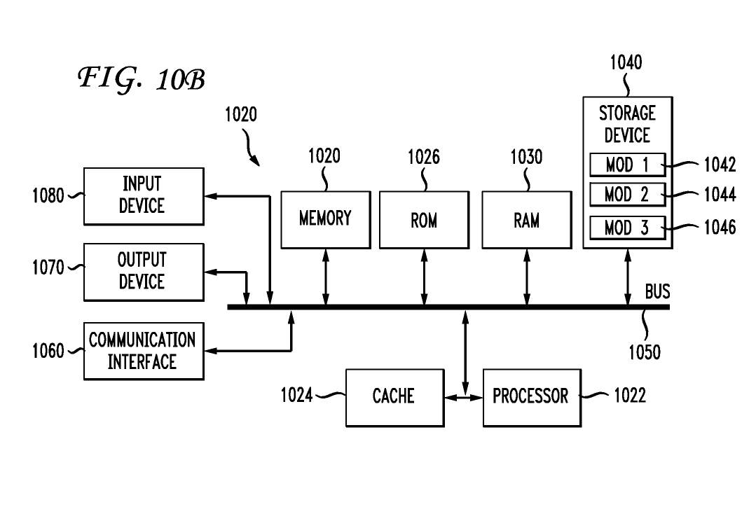

FIG. 10A illustrates a system 1000 that includes a laser 1002, which can be a femtosecond laser or any other type of laser, with a control system 1004. The system shown in FIG. 10B can also be included as appropriate into laser 1002 and/or control system 1004, or in any other system disclosed herein where basic computer control mechanisms are utilized. The computer-guided approach enables the ability to perform precision small capsulotomy as well as the ability to perform in-the-bag lens fragmentation for endocapsular lens removal.

An eye 1000 is positioned and the laser is programmed using a control system 1004 to cut 1006 at least one off-center hole into the anterior portion of the capsular bag 116. The particular settings of the laser 1002 are not material to the present disclosure. In other words, such parameters as focus length, wavelength, duration, number of pulses, etc. can be chosen by an operator to insure an accurate and smooth surface for openings 502, 504.

The laser system 1002, 1004 creates photo-induced plasma 1008 along a vertical line and having a focal point which interacts with the material of the capsular bag 116 and thus cuts the capsular bag 116 at the location of the focal point and plasma. The system 1002, 1004 creates a pattern of focal points simultaneously or sequentially and at different depths (represented by feature 1016) in order to make all the incisions 1010, 1012 and 1014 which yield the openings 502, 504. Shown as feature 1008 is essentially a column of plasma which is utilized to make the appropriate incisions.

The shape of the hole or holes 502, 504, 506 can be any particular shape. While circular is preferable, the number of incisions, the particular size and shape can vary. For example, the holes may be elliptical, square, rectangular or an arbitrary shape. The shape, number of and exact position of the hole(s) may be chosen based on several different parameters, including medical conditions of the patient and desired post-operative biomechanical stretch properties of the capsule. For example, the landscape of the anterior capsule 116 may be analyzed using optical cohesive tomography (OCT) or other imaging systems such as Scheimpful photography, ultrasound or range-finding, in order to select the particular positions and shape used for this capsulotomy. Further, the shape may be chosen based on the particular intraocular lens that this going to be implanted so that the maximum accommodation can result. It is known in the art that people with particular health conditions such as diabetes have different stretch capsular properties. Therefore, the custom capsulotomy envisioned herein can include the choice of shape, size and location of incisions to be based in part on not only an analysis of the capsule 116 but also based on known medical conditions that can affect tensile properties. In so doing, the system and method disclosed herein can be tailored to preserve the maximum or a preferred level of accommodation for each patient. In such a case, the size, location and/or number of incisions can be chosen also based on a predictive model in which the known parameters may indicate what tensile strength might be like in 5 or 10 years although the current OCT or other imaging system analysis does not or cannot indicate or provide a predictive value.

For example, a particular intraocular lens may only be able to be inserted through an elliptically shaped opening in the capsular bag. The control system 1004 can be any type of computer controller software and hardware combination that is capable of selecting and controlling particular scanning parameters in laser firing. Such components may be circuit boards that interface with an OCT scanner, and the focusing device 1002 that directs the laser beam(s) in the Z direction to a particular point or a column. The control system 1004 may contain a particular program which can be used to direct the laser through a number of laser shop patterns and may be able to also be used to measure the position of optical surfaces within the eye such as the portions of the lens such as the anterior portions of the lens, corneal surfaces or other components such as the crystalline lens cataract. Furthermore, the control system 1004 may be used to control a split scanned laser system in order to be able to study and obtain data on the structure of the capsular bag in order to make decisions regarding positioning, size and shape of incision 502 and 504.

The system 1002, 1004, having received data regarding the position of the crystalline lens, the surfaces of the cornea, including the position of the apex of the lens in relation to the laser system and so forth, are utilized in such a way as to enable the laser 1002 to produce incisions in the anterior lens capsule 106 that maintain its accommodation and tensile features. The laser delivery disclosed herein results in precisely determining highly reproducible shaped cuts in patterns as disclosed herein. Again, the particular position of the incisions and their shape may vary from person-to-person based on a number of factors including lens geometry, capsular bag geometry, corneal geometry, type of intraocular lens to be implanted and so forth. The particular manner in which cuts 502 and 504 are made may vary. For example, the particular manner in which the cuts are made may utilize what is disclosed in Frey et al., Publication No. US 2011/0022036 A1, incorporated herein by reference. For example, the laser may cut a hole 502 using a first pattern positioned in a first area of the anterior capsular lens of the eye and having a Z direction sweep range of less than 15 micrometers (μm) and a second patterned position in a second area of the anterior capsular lens of the eye. The second area can be anterior to the first area and the second pattern having a Z direction sweep of range less than about 15 micrometers. This first pattern and the second pattern overlap in the XY dimension. Thus, the additional feature disclosed herein is to perform a capsulotomy having at least one opening that does not overlap in the XY dimension with another opening but rather differs in the XY dimension. One of the openings is for a phacoemulsification device as well for irrigation and aspiration.

The femtosecond laser can also provide pretreatment with fragmentation of the lens prior to aspiration.

FIG. 10B illustrates an example basic computing device which can be utilized in a control system 1004 for a laser 1004 or as part of an overall laser system 1004. With reference to FIG. 10B, an exemplary system 1020 includes a general-purpose computing device 1020, including a processing unit (CPU or processor) 1022 and a system bus 1050 that couples various system components including the system memory 1026 such as read only memory (ROM) 1028 and random access memory (RAM) 1030 to the processor 1022. Other particular designs for control systems for providing a computer-guided laser system for performing automated, non-manual capsulotomies and in-the-bag lens fragmentation are contemplated. The system 1020 can include a cache 1024 of high speed memory connected directly with, in close proximity to, or integrated as part of the processor 1022. The system 1020 copies data from the memory 1026 and/or the storage device 1040 to the cache 1024 for quick access by the processor 1022. In this way, the cache provides a performance boost that avoids processor 1022 delays while waiting for data. These and other modules can control or be configured to control the processor 1022 to perform various actions. Other system memory 1026 may be available for use as well. The memory 1026 can include multiple different types of memory with different performance characteristics. It can be appreciated that the disclosure may operate on a computing device 1020 with more than one processor 1022 or on a group or cluster of computing devices networked together to provide greater processing capability. The processor 1022 can include any general purpose processor and a hardware module or software module, such as module 1 1042, module 2 1044, and module 3 1046 stored in storage device 1040, configured to control the processor 1022 as well as a special-purpose processor where software instructions are incorporated into the actual processor design. The processor 1022 may essentially be a completely self-contained computing system, containing multiple cores or processors, a bus, memory controller, cache, etc. A multi-core processor may be symmetric or asymmetric.

The system bus 1050 may be any of several types of bus structures including a memory bus or memory controller, a peripheral bus, and a local bus using any of a variety of bus architectures. A basic input/output (BIOS) stored in ROM 1028 or the like, may provide the basic routine that helps to transfer information between elements within the computing device 1020, such as during start-up. The computing device 1020 further includes storage devices 1040 such as a hard disk drive, a magnetic disk drive, an optical disk drive, tape drive or the like. The storage device 1040 can include software modules 1042, 1044, 1046 for controlling the processor 1022. Other hardware or software modules are contemplated. The storage device 1040 is connected to the system bus 1050 by a drive interface. The drives and the associated computer readable storage media provide nonvolatile storage of computer readable instructions, data structures, program modules and other data for the computing device 1020. In one aspect, a hardware module that performs a particular function includes the software component stored in a non-transitory computer-readable medium in connection with the necessary hardware components, such as the processor 1022, bus 1050, display 1070, and so forth, to carry out the function. The basic components are known to those of skill in the art and appropriate variations are contemplated depending on the type of device, such as whether the device 1020 is a small, handheld computing device, a desktop computer, or a computer server.

Although the exemplary embodiment described herein employs the hard disk 1040, it should be appreciated by those skilled in the art that other types of computer readable media which can store data that are accessible by a computer, such as magnetic cassettes, flash memory cards, digital versatile disks, cartridges, random access memories (RAMs) 1030, read only memory (ROM) 1028, a cable or wireless signal containing a bit stream and the like, may also be used in the exemplary operating environment. Non-transitory computer-readable storage media expressly exclude media such as energy, carrier signals, electromagnetic waves, and signals per se.

To enable user interaction with the computing device 1020, an input device 1080 represents any number of input mechanisms, such as a microphone for speech, a touch-sensitive screen for gesture or graphical input, keyboard, mouse, motion input, speech and so forth. An output device 1070 can also be one or more of a number of output mechanisms known to those of skill in the art. In some instances, multimodal systems enable a user to provide multiple types of input, sometimes simultaneous, to communicate with the computing device 1020. The communications interface 1060 generally governs and manages the user input and system output. There is no restriction on operating on any particular hardware arrangement and therefore the basic features here may easily be substituted for improved hardware or firmware arrangements as they are developed.

For clarity of explanation, the illustrative system embodiment is presented as including individual functional blocks including functional blocks labeled as a “processor” or processor 1022. The functions these blocks represent may be provided through the use of either shared or dedicated hardware, including, but not limited to, hardware capable of executing software and hardware, such as a processor 1022, that is purpose-built to operate as an equivalent to software executing on a general purpose processor. For example the functions of one or more processors presented in FIG. 1 may be provided by a single shared processor or multiple processors. (Use of the term “processor” should not be construed to refer exclusively to hardware capable of executing software.) Illustrative embodiments may include microprocessor and/or digital signal processor (DSP) hardware, read-only memory (ROM) 1028 for storing software performing the operations discussed below, and random access memory (RAM) 150 for storing results. Very large scale integration (VLSI) hardware embodiments, as well as custom VLSI circuitry in combination with a general purpose DSP circuit, may also be provided.

The logical operations of the various embodiments are implemented as: (1) a sequence of computer implemented steps, operations, or procedures running on a programmable circuit within a general use computer, (2) a sequence of computer implemented steps, operations, or procedures running on a specific-use programmable circuit; and/or (3) interconnected machine modules or program engines within the programmable circuits. The system 1020 shown in FIG. 1 can practice all or part of the recited methods, can be a part of the recited systems, and/or can operate according to instructions in the recited non-transitory computer-readable storage media. Such logical operations can be implemented as modules configured to control the processor 120 to perform particular functions according to the programming of the module. For example, FIG. 1 illustrates three modules Mod1 1042, Mod2 1044 and Mod3 1046 which are modules configured to control the processor 1022. These modules may be stored on the storage device 1040 and loaded into RAM 1030 or memory 1026 at runtime or may be stored as would be known in the art in other computer-readable memory locations.

### Third Embodiment

### Instrumentation

As noted above, the incision approach disclosed herein with minimally invasive capsulotomy renders current instruments unusable because they cannot be manipulated given the small incisions used. The existing opthalmic instrumentation is too rigid and does not allow for directional flexing or extension when inside the eye. Existing conventional intraocular instruments are either advanced, retracted or pivoted entirely at the intraocular entry-point. This deficiency limits the ability of the instruments to operate within the eye especially where it becomes necessary to change the angulation of the instrument tip beyond the corneal, limbal or sclera entry-point. Such movement is not possible given the position and size of the incision(s) disclosed herein. The instruments disclosed herein include one or more probes such as a phaco fragmentation probe, an irrigation probe, an aspiration probe and a capsule polishing probe, or any combination of these probes, that has a flexible and/or extendible tip to allow for intraocular directional angulation and extension beyond the incision point entry into the anterior capsule. One example of technology that can be utilized is shown in U.S. Pat. No. 5,217,465, incorporated herein by reference. The '465 patent shows how a sterrable aspiration tip can be utilized for accessing different areas in the eye. The technology disclosed in this case would be modified for use in the presently disclosed procedures including a smaller incision at a particular location in the capsule.

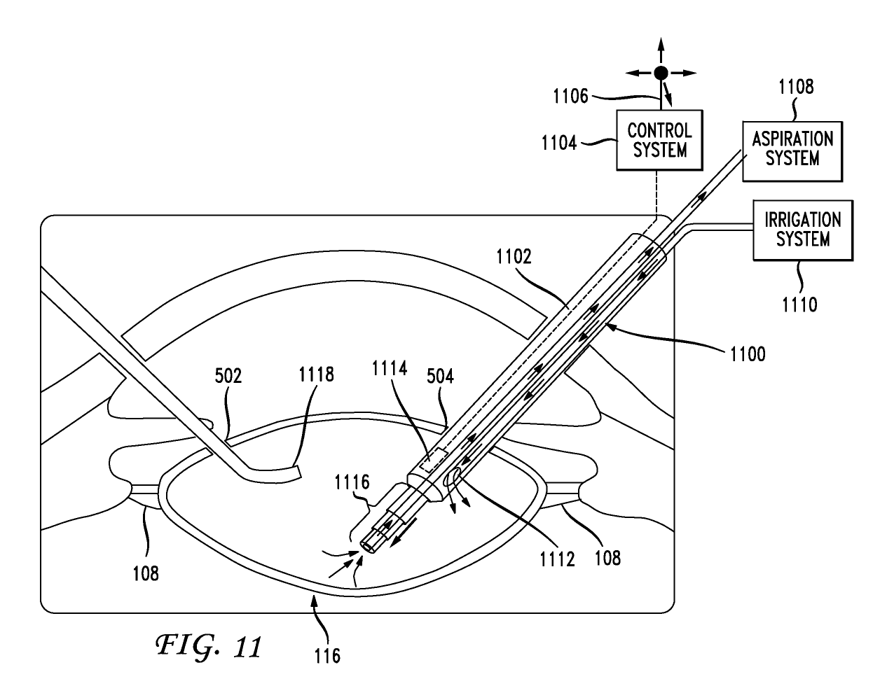

The third embodiment relates to new instrumentation tailored to the novel surgical procedure disclosed herein. An exemplary instrument 1100 is shown in FIG. 11. The instrument 1100 incorporates components to perform various functions that are performed as part of cataract surgery, including an aspiration system 1108, an irrigation system 1110 and a control system 1106 for manipulating a tip portion within a region within the capsular bag 116 a minimally invasive, flexible and/or extendable ophthalmic surgical tool. A neck portion 1102 of the instrument 1100 has a tip end which is inserted through an incision in the cornea and the incision 504 in the capsular bag 116, and a control end which can communicate data and/or material such as lens material or fluid. The neck portion 1102 has an irrigation system 1110 communicating via a channel (not shown) with an opening 1112 which introduces fluid from the irrigation system 1110 into the capsular bag 116 to monitor and maintain pressure as is known in the art. A second channel (not shown) connects an opening at the tip end of a steerable and an extendable tip 1116 and an aspiration system 1108 which communicates lens material and fluid from the chamber defined by the capsular bag 116 to the aspiration system 1108. The tip 1116 can be extended via a telescoping mechanism in which at least one longitudinal component slides within or alongside another longitudinal component or another extension mechanism. The tip 1116 is also steerable through manual movement via 1106 or robotic control. An example steerable aspirator is shown in U.S. Pat. No. 5,217,465, incorporated herein by reference.

A flexible and extendable tip portion 1116 connects to the tip end of the neck portion 1102. A control mechanism 1114 is in communication with a control system 1104 which include, by way of example, a movable member 1106 which a surgeon can use to control the movement the tip portion 1116 within the capsular bag by exending and/or moving laterally the tip 1116 to irrigate and/or aspirate lens material. For example, while generally holding the neck portion 1102 still, the surgeon can move the control member 1106 and have those movements translated or transferred via a module 1114 (which can be electromechanical, nano-technology, etc.) to the flexible and/or extendible tip 1116 to enable one or more of the steps of irrigating, aspirating, chopping and polishing to occur within the chamber defined by the capsular bag 116. The movement occurs from a pivot point beyond the incision entry point into the capsular bag 116. A tip control mechanism is connected to the flexible and extendable tip 1116 and also connected to a user control system 1104. In one aspect, the tip 1116 also includes phacoemulsification (phaco) capability in which the tool 1110 includes an ultrasonic feature. The tip 1116 in this aspect is equipped with a titanium or a steel tip which vibrates at an ultrasonic frequency in the range of 40 kHz or other appropriate frequency. When the tip 1116 comes in contact with the lens 102, the lens is emulsified resulting in lens material which can be aspirated out of the capsular bag. Tool 1118 can also be utilized for phacoemulsification.

The steerable, flexible probe can be a spiral cut probe, a coiled-based design or a two-element hinge probe and can be made of a metal such as steel, NItinol, or other material. In one aspect, the tip 1116 can be controlled by at least one or two pull wires or lines that connect the control mechanism 1114 with the control system 1104. In another aspect, near the tip 1116 of the instrumentation 1100, an internal tube made of plastic or other material can be added (not shown) for the purpose of keeping the fluid/vacuum from escaping through the spiral cut, coil or other portion of the probe. The structure and design of the interal tube can depend on the other structure used to provide the steerable and extendible tip 1116 functionality.

An aspiration opening is positioned generally at the end of the steerable, flexible and extendable tip portion 1116, which aspirates lens material after fragmentation through the second channel to the aspiration system 1108. The movable member 1106 is connected to the user control such that user movement of the movable member 1106 instructs the control system 1104 to cause one of lateral movement and extension or contraction of the flexible and extendable tip portion 1116. The size of the neck portion 1102 is preferably less than approximately 3 mm in order for it to pass through both a cornea incision and an anterior capsular incision 504. FIG. 11 also illustrates a secondary incision 502 including an instrument 1118 which can be used for chopping or other purposes in the procedure.

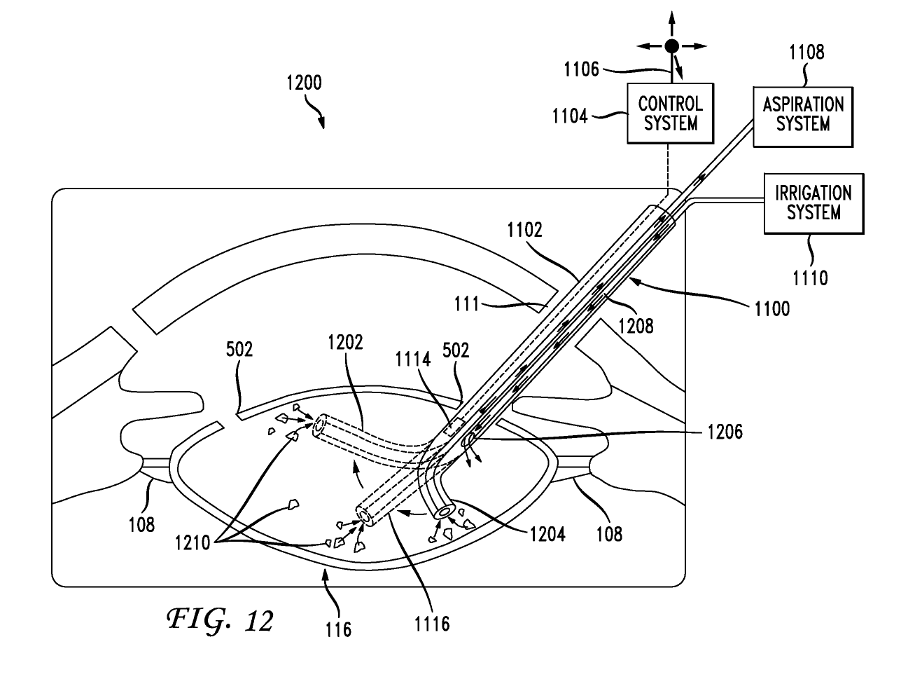

FIG. 12 illustrates instrumentation 1200 similar to that of FIG. 11 but it further illustrates the flexibility of the end tip 1116. Feature 1202 shows the tip 1116 in a position angled to one side as controlled by the member 1106 through the control system 1104 and communicated (mechanically via thin pull wires or electronically) to the local module 1114 to render the final movement of the tip portion 1116. Feature 1204 also shows the tip end 1116 moved to a different position within the capsular bag 116 as controlled by the user via member 1106 and control system 1004. Lens material 121, which is distributed throughout the chamber, can therefore be more easily aspirated as the surgeon moves the tip member 1116 around within the capsular bag 116. An example irrigation opening 1206 near the tip end of the neck portion 1102 of the instrument 1100 is shown. The channel 1208 that communicates (fragmented) lens material from the tip end 1116 through the channel 1208 to the aspiration system 1108 is also flexibility in the tip portion 1116 such that the tip end 1116, having the aspiration channel 1208, can extend and flex from side to side in such a way as to maintain the ability to aspirate through the tip end in each position. In this manner, while the neck portion 1102 of the instrument 1100 is in a generally fixed position through the opening 111 in the cornea and opening 504 in the anterior capsule, the surgeon is able to irrigate and aspirate the entire volume inside the capsular bag 116. Tip 1116 can also include an ultrasonic component which can be controlled. Thus, the steerable and extendible tip 1116 can include a phaco tip that can vibrate for emulsification. The vibration component can be included in the extendible and steerable portion to reach out and emulsify portions of the lens that may still need processing for aspiration.

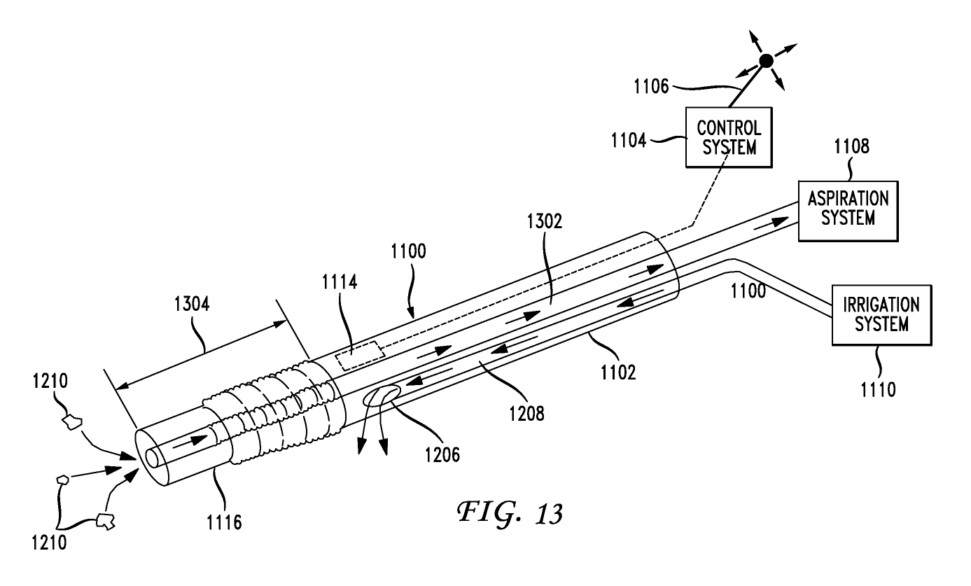

FIG. 13 illustrates in more detail the neck portion 1100 of an instrument with the tip control system 1104, control member 1106, and the control module 1114. Tip end 1116 is shown in a partial extended position as directed by the control system 1104. The opening 1206 is connected via a channel 1208 to the irrigation system 1110. Lens material 1210 and fluid in the chamber of the capsular bag 116 can flow through the opening in the tip portion 1116, through a channel 1302 and to the aspiration system 1108. Other components are shown such as the aspiration system 1108 connected via channel 1302 to the end of the tip portion 1116 which is used to aspirate lens material 1210.

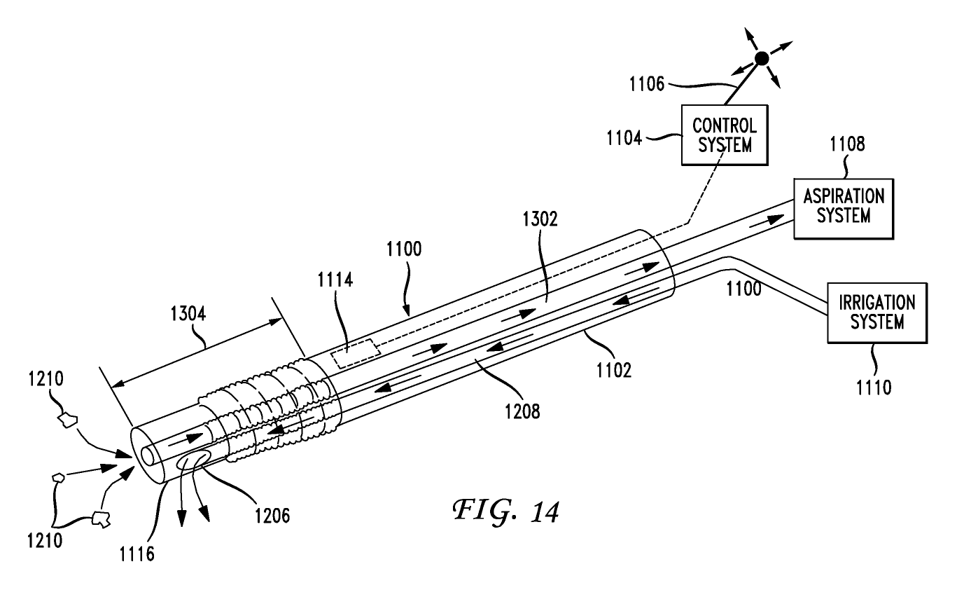

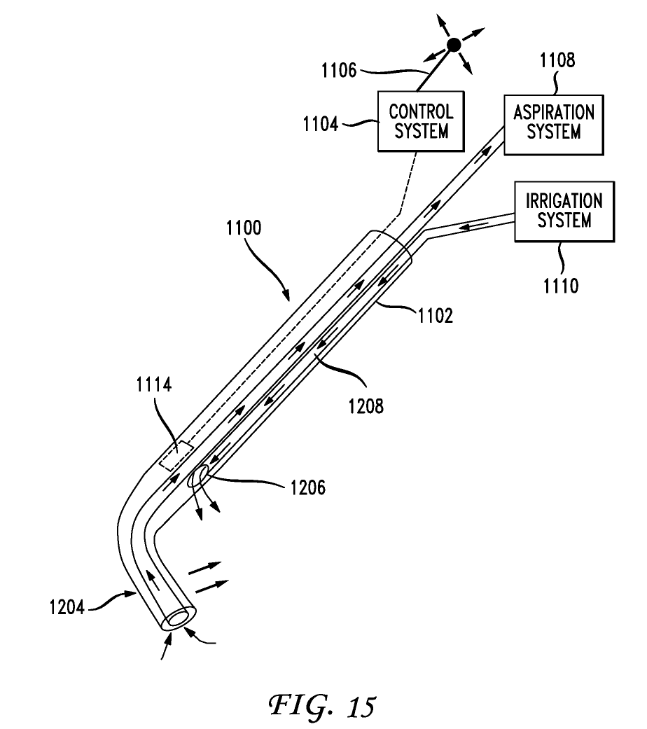

FIG. 14 shows a similar visual with respect to FIG. 13 but with the opening 1206 being positioned at an end portion of the tip 1116. Thus, the irrigation can provide fluid into the chamber at a controllable position of the instrument. FIG. 15 illustrates the instrument 1100 having the neck portion 1102, a channel 1208 communicating an irrigation opening 1206 with the irrigation system 1110. The control module 1114 communicates with the control system 1104 such that the tip end 1204 can be moved in a side to side motion such that lens material and fluid can flow into the channel that communicates the opening in the end tip 1204 with the aspiration 1108.

### Forth Embodiment

### Robotic System

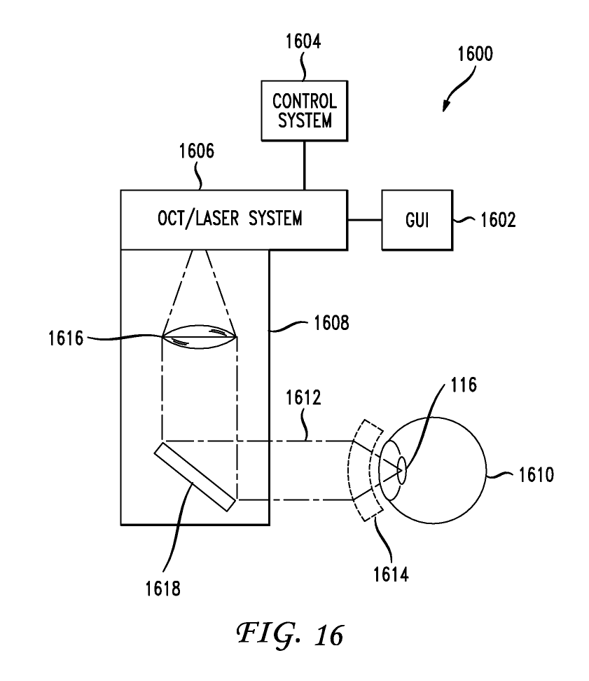

This disclosure next turns to a robotic system embodiment. FIG. 16 illustrates a system 1600 which includes a mechanism for evaluating the 3D environment of the eye through a combination of approaches. Optical coherence tomography (OCT), ultrasound, or other imaging system as well as a laser system such as a femtosecond laser can be used to sense the structure of the various eye components. Sensors are used to identify the general environment in which the robot will be programmed to perform the various steps of performing a capsulotomy, irrigation, aspiration, polishing and so forth as part of the steps performing cataract surgery. The system 1600 can be used to locate and define the surface of the capsular bag 116 (and more specifically the anterior capsule) such that the laser 1612 beam will be focused in the appropriate portions of the anterior lens capsule at the appropriate points to create the desired cuts. Any type of imaging modality may be used to determine the location and characteristics of the capsular bag 116 as well as the thickness and location of the lens within the capsular bag 116. The data can include identification of the tensile strength structure across the anterior region of the capsular bag 116. The system 1600 obtains this data and can include 2D and/or 3D imaging and patterning to give the user via a GUI 1602 a visual image of the volume in which the surgery will be performed. Laser focusing may also be accomplished using one or more methods such as direct observation of an aiming beam, OCT, ultrasound or any other known ophthalmic or medical imaging approach or any such combinations.

In another aspect, the robotic system could mechanically perform the tension sparing capsulotomy without using a laser. In this scenario, a plasma knife, mechanical knife or other surgical tools could be used to accomplish the procedure as well.

A simple linear scan using the system 1606, 1608 across the capsular bag 116 and lens 102 can produce data about the space which will be utilized for both the incision in the anterior capsule as well as the endocapsular fragmentation, dissolution, liquefaction, etc. The scan can provide information about an axial location of the anterior and posterior lens capsule, tensile structure and characteristics of the capsular bag 116, the boundaries of the cataract nucleus, as well as the depth of the anterior chamber. The information is loaded into the control system 1604 and utilized to program and control the subsequent laser assisted surgical procedure.

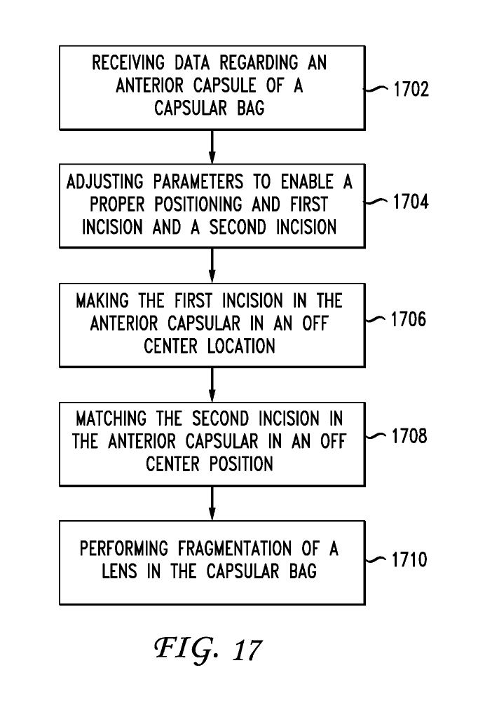

The information can be used to determine a wide variety of parameters related to the procedure such as, the appropriate positioning and size of incisions 502 and 504 in the anterior capsule 106, the shape of the incisions 502, 504 how and in what manner to pattern a fragmentation of the lens. The data can also enable programming based on what are the upper and lower axial limits of the focal planes for cutting the lens capsule and segmentation of the lens cortex and nucleus, as well as the thickness of the lens capsule. Furthermore, as shall be shown later with FIG. 17, the information obtained from the 3D scan will be used when the robotic procedure inserts the improved instrument for the purpose of irrigation and aspiration.

Other components of the imaging/guidance/laser system include optic lenses 1618 and 1616 which are not introduced for the purpose of limiting the present disclosure but to give some basic information regarding the optics which can be used according to this disclosure. Any optical structure and functioning of a laser that is appropriate for cataract surgery may be employed in the present disclosure.

Feature 1614 illustrates an optional ophthalmic lens that can be used to focus the beam 1612 into the patient's eye 1610. This lens 1614 can also be used in order to gather the scan data for use in mapping the 3D position of the capsular bag 116 and its associated components.

Next, following the obtaining of the information from the scanning system, the OCT laser system 1606 can utilize that information and perform robotically the first steps of the surgical procedure.

FIG. 17 illustrates the steps that can be performed either robotically as in this current embodiment or partially manually and partially aided by a programmed laser or other device. As is shown in FIG. 17, the system can receive data regarding an anterior capsule of a capsular bag, and other data regarding the lens, lens depth, information regarding the tensile properties and structure over the region of the anterior capsule, and so forth (1702). Utilizing the information that is received, the system adjusts parameters to enable a proper positioning of at least one incision and preferably a first incision and a second incision within the anterior capsule of the capsular bag (1704).

The system makes the first incision in the anterior capsule in an off-center location (1706). The system optionally makes a second incision and the anterior capsule in an off-center position (1708). As noted above, another optional approach is to utilize a small incision (approximately equal to or less than 3 mm) in a central region of the anterior capsule. Such an incision could be used where it is a preferable choice given the tension sparing capsular forces and how they are structure across the anterior capsule. Thus, one of the incisions could be centrally located as well. Next, the system 1606 performs fragmentation of the lens in the capsular bag 1710. At this point, either the robotic system or the surgeon removes the small portion of the anterior capsule that is cut out from at least one of the first incision and the second incision leaving one or two openings in the anterior capsule. It is noted that because of the characteristics of the volume within the capsular bag are known because of the scanning step, that the laser system can accurately perform fragmentation of the hardened lens such that it can easily be aspirated. In one aspect, the system 1604, 1606 because it knows the position of the incisions 502, 504, 506 can fragment the lens in a pattern which may be more advantageous for the aspiration step. For example, the system 1604, 1606 may cause the size of the lens material fragments that are near incisions 502, 504 to be smaller or bigger than other relative pieces of the lens material which can ease in the aspiration process. The size and shape of lens material that is fragmented can also be chosen based on a shape of or position of one or more incisions 502, 504. Additionally, the cross sectional shape of a surgical instrument such as neck 1102 can be chosen based on the phase of the incision through which the instrument must pass.

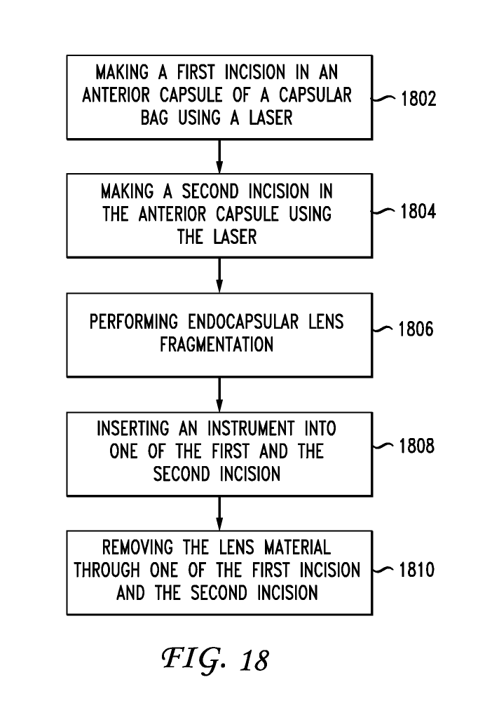

FIG. 18 illustrates another method embodiment. The method includes making a first incision in the anterior capsule of the capsular bag 116 using a femtosecond laser (1802). Any appropriate laser is also contemplated. The first incision is made off-center from a central point in the anterior capsular. The first incision has a diameter of approximately 3 mm or less and can be any shape. The method includes making a second incision in the anterior capsule using the laser (1804) and performing an external lens endocapsular lens fragmentation of the lens contained within the capsular bag (1806). The second incision is approximately 2.5 mm in diameter or less and is also off center. Included in this method is an optional step of scanning the structure of the eye including the physical properties and tensile characteristics of the capsular bag 116 and particularly the anterior surface 106 to yield data. At least one of the size, location and shape of at least one of the first and second incision can be implemented based on the data. Fragmentation in this case means any pre-treatment of the lens such as dissolution, phacoemulsification, liquefaction, etc. Preferably, the capsulotomy and the in-the-bag fragmentation, are performed non-manually and computer-guided by a laser or other device. For example, a laser could automatically perform the capsulotomy and a phacoemulsification system could emulsify the lens (as opposed to fragmentation by a laser) in a computer-aided manner.

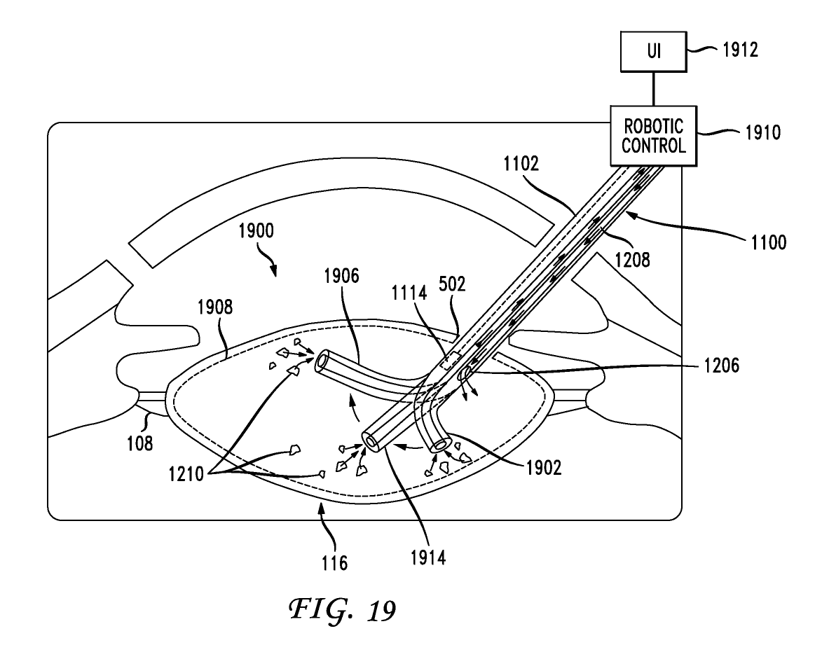

Either robotically or manually, the method includes inserting an instrument into one of the first incision and the second incision (1808) and removing the lens material from one of the first incision and the second incision (1810). Based on the data related to the scan discussed above, an internal volume boundary within the capsular bag can be established which defines a space in which an instrument can safely roam to irrigate and/or aspirate without damaging an interior surface of the capsular bag 116. The boundary can also be used to guide polishing of surfaces where necessary inside the capsular bag 116. The data utilized in the scan is discussed further with respect to FIG. 19 below. An intraocular lens is inserted into the capsular bag after aspiration is complete. Using the techniques disclosed herein can result in maintaining at least 2 to 3 diopters of accommodation even from a monofocal intraocular lens.

FIG. 19 illustrates the robotic control of this embodiment. This embodiment provides a system that safely enables intraocular instruments such as phaco probes, irrigation probes, aspiration probes, suction probes, and any other instrument, to be positioned, angulated, and extended within the eye easily from a pivot point within the chamber defined by the capsular bag 116. The instrumentation and robotic control increases the safety and predictability of cataract surgery and can maintain accommodation for the patient following the surgery because the tensile structure across the anterior capsule 106 can be maintained. Because the incisions in the anterior capsule 106 are smaller and positioned off center, the margin of error is smaller and thus a robotic approach can increase the accuracy and success of cataract surgery. The tip portion of the instrument 1100 is primarily shown as an aspiration probe but it represents all types of probes which can be used including, but not limited to, a phaco fragmentation probe, an irrigation probe, an aspiration probe, and a capsule polishing probe.

As is shown in FIG. 19, the capsular bag 116 has an incision 504. As noted above, another incision (not shown) can also be provided for a “chopper,” “irrigating chopper,” or other instrument. A neck portion 1102 of an instrument 1100 is inserted through the small incision 504 into the chamber defined by the capsular bag 116. A robotic control 1910 is connected to the instrument 1100 which includes an optional user interface 1912 that enables the surgeon in some cases to overrule the robotic controls. The system shown in FIG. 10B as well as the computer readable medium discussed below can be included as part of the hardware for controlling the robotic control system 1910.

Outline 1908 illustrates the volume boundary from the scan discussed above. The robotic control 1910 can operate within parameters defined by the volume boundary 1908 to prevent the movement of the tip portion of the instrument 1100 from damaging the inner surface of the capsular bag 116. The robotic control 1910 controls the movement of the tip portion into various positions within the capsular bag 116 while always maintaining a minimal distance from the inner surface of the capsular bag 116 as defined by the boundary 1908. By way of example, when the tip portion is in position 1902, the aspiration process can occur far enough away from a posterior surface 104 of the capsular bag 116 that could be damaging. As is shown in FIG. 19, the tip end of the instrument 1100 allows for intraocular directional angulation beyond the incision entry point 504.

Position 1904 of the tip portion of the instrument 1100 illustrates the tip extended to a particular position in order to aspirate lens material 1210, again in such ways that it does not approach too closely to the anterior portion 104 of the capsular bag 116. The extension can occur via a telescoping structure (i.e., where longitudinal elements slide over each other for either extending or retracting the telescoping structure) or other types of extension mechanism. Position 1906 illustrates the flexible nature of the tip of instrument 1100 also performing aspiration in that portion of the inner chamber of the capsular bag 116. Opening 1206 also shows one example position of an opening for irrigation into the eye in order to maintain pressurization, etc. The point of flexion or extension within

Utilizing robotic control 1910, the system can automatically sweep the interior chamber of the capsular bag 116 in a pattern or dynamic method such that each region is aspirated and all of the lens material 1210 can be retrieved. The probe 1902, 1904, 1906 represents a flexible, telescoping and/or extendable probe which can be one or more of an irrigation probe, an aspiration probe, a combination irrigation/aspiration probe as is shown, and a phaco probe.

In one aspect, the robotic control 1910 simply performs a preplanned sweep of the entire volume assuming that the vast majority of the lens material will be aspirated appropriately. Following a controlled sweep, the surgeon can then manually using an interface 1912 insure that the eye is fully cleansed of lens material in preparation for receiving the new intraocular lens. In another aspect, the system can have feedback sensors within the tip portion or within the system 1910 which can help to identify where a particular lens material 1210 exists. Based on the feedback (such as variations in pressure that is felt at the tip of the aspirating instrument), the system can seek after and specifically identify and aspirate individual fragments of the lens material 1210.

In another aspect, while the robotic control 1910 can prevent the tip portion from scratching or using its vacuum from scratching or damaging the interior surface of the capsular bag 116, the system could enable a user interface 1912 to overrule the robotic control 1910 and have a manual control which could then be used to make any final aspiration or other necessary steps in the process of the surgery.

For example, while the positioning defined by parameter 1908 may be a safe distance away from the inner surface of the capsular bag 116 in order to avoid damage, the defined distance can prevent the aspiration instrument 1100 from retrieving all of the lens material 1210. In that case, the user interface 1912 may enable a surgeon to override the boundary in order to manipulate the tip portion of the instrument into the space between the parameter 1908 and the capsular bag 116. This overriding feature can enable the surgeon to make any corrections or further cleaning that needs to occur.

Next, the robotic control can also use the volume information in order to perform an appropriate polishing if such polishing is desirable. Feature 1914 of FIG. 19 represents a surface of the tip portion that can be utilized for polishing an inner surface of the capsular bag 116. The polishing structure can be a separate probe or shown as part of a probe having another function. The polishing probe can be flexible and curved as well. The robotic control, using the information about the boundary 1908, and the depth, nature and characteristics of the capsular bag 116, could, following the aspiration step, perform polishing on either the anterior portion 104 of the capsular bag 116 or the anterior portion of the capsular bag 106. The polishing prevents the opacification of the anterior capsule which is not covering the central optical access. Therefore, the robotic control 1910 can utilize a capsular polisher or vacuum clean up of proliferating cells that are associated with the capsular bag 116 that may have been generated by virtue of the surgical procedure.

### Fifth Embodiment

### Intraocular Lens Design

Given the smaller size of the incisions 502, 504, 506, according to this disclosure, there is a need for improved lens design for foldable intraocular lenses that can safely be inserted through both the opening in the cornea and the opening in the anterior capsular 106. Preferably, the intraocular lens is a single soft piece of monofocal material that is inserted into the capsular bag 116 via a small injector. Example embodiments include the Raysert injector which can be used in an incision around 1.8 mm with a C-Flex lens. A Blyemix injector from the Zeiss company can also be used through an incision of 1.8 mm with a CT Spheric intraocular lens. A B&L injector also can be used which can operate through an incision of 1.8 mm with an Akreus intraocular lens. These and other lenses can be utilized to finalize the cataract surgery.

With respect to tension sparing capsulotomy, there are specific IOL designs which are intended to maximize the preserved accommodative capsular biomechanics. Currently, conventional IOLs are either uniplanar or back-vaulting to prevent protrusion and capture of the optic in the large central capsulotomy. With tension sparing capsulotomy, forward vaulting IOL designs are best suited to capture the accommodative forces without any concern for IOL capture/escape. Also, four-point IOL designs with forward vault are likely to offer accommodating optical advantages with tension sparing capsulotomy over traditional capsulotomy. In addition, IOLs designs whereby the haptics are stiffer peripherally and softer (more flexible) centrally will provide higher accommodating potential. Furthermore, an IOL embodiment optimized for a tensile sparing capsolotomy can have a much smaller central optic than current designs where central optic is at 5.5 mm or more. With smaller central optics (between 2 and 5.5 mm) the longer haptics will allow more movement of the central optic for accommodation.

Some of the features of IOLs that need to be tailored to the resulting anterior capsular environment as described herein include the ability of a polymer to (1) have viscoelastic qualities amenable to deformation, (2) accurately target emmetropia during disaccommodation and 3) return to its resting shape during accommodation. The accommodating IOL must also produce minimal aberrations through the transition between accommodation and disaccommodation. The basic approaches to modifying the design of IOLs is to (1) change the axial position of a single or dual-optic IOL, (2) change the IOL optic's shape or surfaced curvature and (3) effect a dynamic change in the refractive index or power of a single or dual-optic IOL. There may also be benefits from an additive effect of pseudoaccommodative mechanisms.

Various approaches to adjusting these features of IOL's are disclosed in the article New Accommodating IOLs, by Jay S. Pepose, in Advanced Ocular Care, October 2011, incorporated herein by reference. One IOL called the SmartIOL that is disclosed is made with a unique thermodynamic property that converts it shape from a solid rod (for insertion in a small incision of size 3.0-3.5 mm) to a gel-like lens-shaped polymer at body temperature when inserted. Applying this particular IOL to the present disclosure would require some basic changes. For example, approach using an IOL that changes its thermodynamic properties would be to prepare a number, say 20, of different resulting lens-shaped polymers that have different properties based on a different accommodating tensile structure for a patient following a capsulotomy as disclosed herein. Based on an understanding of the particular available accommodation in the anterior capsule 106, one of the lens designs would be chosen. The diameter of the solid rod of the lens material would be modified to enable insertion into a smaller incision of a size less than 3.0 mm. Once the rod is inserted into the capsular bag, the body temperature would cause the material to transform into the gel-like polymer and take the shape of the lens that has properties that match the accommodative features of the anterior capsule and thus provide improved accommodation after cataract surgery. One or more of the characteristics of a polymer can be modified or adjusted for a particular patient's accommodative ability based on the procedures disclosed herein. Such characteristics include one or more of: the viscoelastic qualities amenable to deformation, the ability to target emmetropia during disaccommodation, and the ability to return to its resting shape during accommodation. The changes in the axial position of a single or dual optic IOL, the refractive index or power of an IOL and a change in the IOL optic's shape or curvature can each be adjusted based on the tensile structure left after the capsulotomy disclosed herein.

Another IOL referenced in the Pepose article incorporated above is the Elenza electroadaptive accommodating IOL. This includes features such as auto-programmable dual ASiCs, dual lithium batteries, an electro-active liquid crystal providing +2.0-2.5 D and an aspheric central optic for far and intermediate vision. This IOL is based on electrical control of the refractive index of a nematic liquid crystal sandwiched between a circular array of photolithographically-defined transparent electrodes. It operates with a high transmission, low voltage, fast response, high-diffraction efficiency and a power failure-safe configuration. There is a monofocal static IOL that has an aspheric central optic for far and intermediate vision. The diffractive liquid crystal is electroactivated for near vision. Microsensors detect physiological changes in light triggered by accommodative effort, and the processors and algorithms control the power sequence.

The additional features that apply to the present disclosure is that the microsensor are modified such that either the particular accommodative ability of eye based on the size and/or location of the incisions in the surgery disclosed herein is taken into account to improve the accuracy of the microsensors. Thus, each patient may have a tailored algorithm for their type of surgery and the tensile structure that remains in the anterior capsule 106. For example, the difference in illumination via miosis for a patient after cataract surgery may be different than the average expected illumination. These changes can be incorporated into the algorithm. Further, the microsensor can also sense tension or movement within the eye instead of or in addition to sensing illumination, which can result in the processor controlling the diffractive liquid crystal to “focus” for near vision, intermediate vision or far vision.

### Sixth Embodiment

### A Computer-Readable Medium

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Those of skill in the art will appreciate that other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The various embodiments described above are provided by way of illustration only and should not be construed to limit the scope of the disclosure. For example, the principles herein can be applied to speech recognition in any situation, but can be particularly useful when the system processes speech from a user in a noisy environment. Those skilled in the art will readily recognize various modifications and changes that may be made to the principles described herein without following the example embodiments and applications illustrated and described herein, and without departing from the spirit and scope of the disclosure.

1. A method of performing a capsulotomy in which each incision is sized and positioned to maintain at least a portion of capsular tensile strength and integrity within a central area of an anterior capsule of a capsular bag, the method comprising:
making an incision in the anterior capsule of the capsular bag, the incision being less than 3.5 mm in diameter, wherein the method comprises making no more than three total, non-overlapping incisions in the anterior capsule such that at least a portion of the central anterior capsule is preserved to maintain tensile strength and accommodative capacity;
performing endocapsular lens fragmentation of a lens in the capsular bag to yield lens material;
inserting an instrument into the incision; and
removing the lens material via the instrument and through the incision.

2. The method of claim 1, wherein the instrument comprises a tip that is one of flexible and extendible to allow intraocular directional angulation within the capsular bag.

3. The method of claim 1, further comprising polishing the anterior capsule.

4. The method of claim 3, further comprising implanting a new lens through the incision.

5. The method of claim 1, wherein the incision is off-center from a center portion of the anterior capsule.

6. The method of claim 1, further comprising making a second incision in the anterior capsule of the capsular bag, wherein the second incision is less than or equal to 3.0 mm in diameter, wherein the first incision and the second incision are positioned off-center from a center portion of the anterior capsule.

7. (canceled)

8. The method of claim 1, wherein making the incision in the anterior capsule of the capsular bag is performed using a laser.

9. The method of claim 8, wherein the laser is a femtosecond laser.

10. The method of claim 1, further comprising irrigating within the capsular bag.

11. The method of claim 1, further comprising polishing a portion of an inner surface of the capsular bag.

12. A method comprising:
making a first incision in an anterior capsule of a capsular bag, the first incision being less than or equal to approximately 3.5 mm in diameter;
making a second incision in the anterior capsule, the second incision being less than or equal to approximately 3.0 mm in diameter, wherein the first incision and the second incision are positioned off-center from a center portion of the anterior capsule;
performing lens fragmentation of a lens in the capsular bag to yield lens material;
inserting a first instrument into the first incision;
inserting a second first instrument into the second incision; and
removing the lens material via one of the first instrument and the second instrument and through one of the first incision and the second incision.

13. The method of claim 12, further comprising:
polishing the anterior capsule; and
implanting a new lens through at least one of the first incision and the second incision.

14. The method of claim 6, further comprising performing pre-treatment of the lens for removal of the lens material through one of the first incision and the second incision.

15. The method of claim 12, wherein one of the first instrument and the second instrument comprises a tip that is one of flexible and extendible to allow intraocular directional angulation within the capsular bag.

16. The method of claim 12, wherein making the first incision and making the second incision are performed using a laser.

17. The method of claim 16, wherein the laser is a femtosecond laser.

18. The method of claim 12, wherein making the first incision and making the second incision result in maintaining at least a portion of capsular tensile strength and integrity across the anterior capsule.

19. The method of claim 1, wherein the at least a portion of capsular tensile strength and integrity within the central 6 mm area of the anterior capsule that is maintained is of at least 2 diopters.

20. The method of claim 12, further comprising:
polishing at least a portion of an interior surface of the capsular bag.

21. A system comprising:
a processor;
a laser; and
a computer readable medium storing instructions, which, when executed by the processor, cause the processor in connection with the laser to perform operations comprising:
making a first incision in an anterior capsule of a capsular bag, the first incision being approximately equal to or less than 3.5 mm in diameter; and
making a second incision in the anterior capsule, the second incision being approximately equal to or less than 3.5 mm in diameter, wherein the first incision and the second incision are positioned to maintain at least a portion of capsular tensile strength and integrity across the anterior capsule.

22. The system of claim 21, wherein at least one of the first incision and the second incision is positioned off center from a center portion of the anterior capsule.

23. A device comprising:
a neck portion having a tip end and a control end, the neck portion having a first channel for irrigating an eye during a surgery and a second channel for aspirating material from the eye during the surgery;
a flexible and extendable tip portion connected to the tip end of the neck portion;
a tip portion control mechanism connected to the flexible and extendable tip and connected to a user control system;
an irrigation opening positioned generally at the tip portion and connected through the first channel with an irrigation system that causes material to flow through the first channel and through the irrigation opening into the eye;
an aspiration opening, positioned on the flexible and extendable tip portion, which aspirates lens material from the eye through the second channel to an aspiration system; and
a movable member connected to the user control such that user movement of the movable member causes the control medium to perform one of lateral movement and extension or contraction of the flexible and extendable tip portion from a point beyond an incision entry point in the eye.

24. A method comprising:
via an automated, computer-guided system:
making an incision in an anterior capsule of a capsular bag, the incision being less than approximately 3.5 mm in diameter, wherein at least one of a size, a position, and a shape of the incision are chosen based on feedback associated with a capsular tensile strength across the anterior capsule; and
performing endocapsular lens fragmentation of a lens in the capsular bag to yield lens material.

25. The method of claim 24, further comprising aspirating the lens material via the incision and inserting an intraocular lens into the capsular bag via the incision.

26. The method of claim 24, wherein the automated, computer-guided system comprises a laser and wherein the position of the incision is off-center in the anterior capsule.

27. The method of claim 24, wherein the automated, computer-guided system is programmed to choose a position for the incision in an off center position if the feedback indicates that the off center position would preserve the capsular tensile strength across the anterior capsule.
